(19) United States
(12) Patent Application Publication
(10) Pub. No.: US 2020/0353147 A1
(43) Pub. Date: Nov. 12, 2020

(54) FLUID PUMPING DEVICE AND BLOOD PURIFYING APPARATUS HAVING THE SAME

(71) Applicant: Jake K. Lee, South Burlington, VT (US)

(72) Inventor: Jake K. Lee, South Burlington, VT (US)

(21) Appl. No.: 16/703,757

(22) Filed: Dec. 4, 2019

Related U.S. Application Data
(60) Provisional application No. 62/845,292, filed on May 8, 2019.

(30) Foreign Application Priority Data
Nov. 4, 2019 (KR) 10-2019-0139273

(51) Int. Cl. A61M 1/26; A61M 1/16

(52) U.S. Cl. CPC A61M 1/267; A61M 1/262; A61M 2205/3331; A61M 2205/3334; A61M 2205/3337; A61M 1/1601

(57) ABSTRACT

Provided is a blood purifying apparatus having a fluid pumping device, which further includes first to sixth chambers each having an internal space, a chamber pressurizing member compressing or expanding the internal spaces of the fluid chambers, a chamber pressurizing member driver driving the chamber pressurizing member, and a flow controller. The chambers are each connected with a first flow tube through which a fluid is provided to the chamber and a second flow tube through which a fluid of the chamber is discharged therefrom. The flow controller controls a flow passage through the flow tubes connected to the first to fourth chambers.

### CROSS-REFERENCE TO RELATED APPLICATIONS

This application claims the benefit of priority to U.S. Provisional Patent Application Ser. No. 62/845,292 filed on May 8, 2019, and claims priority under 35 U.S.C. § 119 to Korean Patent Application No. 10-2019-0139273 filed on Nov. 4, 2019, the entire contents of which are incorporated by reference herein.

### TECHNICAL FIELD

The present invention relates to a fluid pumping device and a blood purifying apparatus having the same, and more particularly to a blood purifying apparatus having the fluid pumping device in which a plurality of fluid chambers are compressed and expanded at the same time using a single chamber pressurizing member to allow both blood and dialysis fluid to flow therethrough, thereby simplifying the overall apparatus, providing easy installation, and reducing the cost for blood purifying treatment.

### BACKGROUND

When there is a kidney dysfunction, water and waste products that have to be discharged out of body accumulate in blood and imbalance of electrolytes in the body occurs. Most commonly performed to improve such a kidney failure symptom, is hemodialysis which is to circulate blood out of body and rid the blood of the accumulated uremic toxin and excess water by a semi-permeable dialysis membrane. Hemodialysis is a method of seeking an electrolyte balance and ridding the body fluid of uremic toxin and excess water, taking advantages of diffusion applied due to the concentration difference and filtration applied due to the pressure difference between blood and dialysis fluid.

Most commonly used of blood purifying filter is the type that is a cylinder-shaped container charged with a bundle of hollow fiber membranes and port-processed at both ends thereof by use of a synthetic resin like polyurethane. It is because the hollow fiber blood purifying filter has excellent mass-transfer efficiency resulting from large effective surface area between blood and dialysis fluid compared to the small size as a whole.

Blood and dialysis fluid each decrease their hydraulic pressure while passing through a blood purifying filter. Since blood and dialysis fluid flow in opposite directions inside the blood purifying filter, a filtration occurs at the proximal part of the blood purifying filter such that water in the blood moves toward dialysis fluid compartment because blood pressure is higher than dialysis fluid pressure, while a backfiltration occurs at the distal part such that water in the dialysis fluid moves toward blood domain for the same reason.

Conventional hemodialysis devices entail a balancing chamber unit which is connected to both the dialysis fluid supply and discharge lines and two or more dialysis fluid pumps while blood is circulated by a separate blood pump. Also, it is indispensable to disinfect the balancing chamber unit and the dialysis fluid pumps on a regular basis. Thus, it is inevitable that the conventional hemodialysis devices become very complex in the structure and complicated for patients to use at home.

### SUMMARY

Provided is a blood purifying apparatus having a fluid pumping device including a plurality of fluid chambers each having an internal space, a chamber pressurizing member compressing or expanding the internal spaces of the fluid chambers, a chamber pressurizing member driver driving the chamber pressurizing member, and a flow controller.

The plurality of fluid chambers may include first to sixth chambers, and each of the first to sixth chambers is connected with a first flow tube through which a fluid is provided to the chamber and a second flow tube through which a fluid of the chamber is discharged therefrom. Here, the flow controller is able to control a flow passage through the flow tubes connected to the first to fourth chambers. In addition, in an embodiment, any three chambers of the first to sixth chambers may be compressed by the chamber pressurizing member while another three chambers are expanded.

The blood purifying apparatus according to an embodiment of the present invention may further include a one-way valve installed on each of the flow tubes connected to the fifth chamber and the sixth chamber to allow a fluid to flow in one direction.

The flow controller according to an embodiment of the present invention may have one or more valve structures, such as (a) a solenoid valve installed on each of the flow tubes through which the flow controller controls a flow to open or block a flow therethrough, (b) an on-off valve installed on each of the flow tubes through which the flow controller controls a flow to open or block a flow therethrough, (c) a pressurizing-type valve having a flow-blocking member compressing a portion of the flow tubes through which the flow controller controls a flow, a flow-blocking wall supporting the flow tubes compressed by the flow-blocking member, and a flow-blocking member driver providing a liner or curved movement force to the flow-blocking member, and (d) a rotating-type valve including a flow control housing having a cylinder-shaped internal space, a flow control rotor having a cylinder shape and disposed inside the internal space of the flow control housing, a plurality of flow control ports each penetrating the flow control housing between the internal space and an outer surface thereof, and a rotor driver driving the flow control rotor.

In addition, when the chambers are compressed or expanded, the flow controller according to an embodiment of the present invention may be able to block a flow through at least a half of the flow tubes through which the flow controller controls a flow.

Disclosed is a blood purifying apparatus which can be easily installed and used by not only medical personnel but also patients (and their family members) due to a novel fluid pumping device which can transfer blood and dialysis fluid simultaneously through a single driver. Since a single driver can transfer both blood an dialysis fluid for the present blood purifying apparatus, cost for the device and treatment both can be substantially decreased and the overall device can be sufficiently miniaturized and light-weighted, rendering the present blood purifying apparatus an optimal alternative for blood purifying treatment in a place out of the clinics or hospitals, such as at home.

### BRIEF DESCRIPTION OF THE DRAWINGS

The accompanying drawings are included to provide a further understanding of the present invention and are incorporated in and constitute a part of this specification. The drawings illustrate exemplary embodiments of the present invention and, together with the description, serve to explain principles of the present invention. In the drawings:

FIG. 1 is a view illustrating a blood purifying apparatus according to an embodiment of the present invention;

FIG. 2 is a view illustrating a blood purifying filter according to an embodiment of the present invention;

FIG. 3 is a view illustrating a fluid pumping device according to an embodiment of the present invention;

FIG. 4 is a view illustrating a blood purifying apparatus according to an embodiment of the present invention;

FIGS. 5 and 6 are views illustrating an operation of a blood purifying apparatus according to an embodiment of the present invention;

FIGS. 7 to 10 are views illustrating a flow controller according to an embodiment of the present invention;

FIG. 11 is a view illustrating a fluid pumping device having one-way valves for a flow controller according to an embodiment of the present invention;

FIG. 12 is a view illustrating a blood purifying apparatus having one-way valves for a flow controller according to an embodiment of the present invention;

FIGS. 13 to 16 are views illustrating an operation of a blood purifying apparatus having one-way valves for a flow controller according to an embodiment of the present invention;

FIG. 17 is a view illustrating a blood purifying apparatus, in which a flow controller controls a flow through a plurality of fluid chambers, according to an embodiment of the present invention;

FIGS. 18 and 19 are views illustrating an operation of a blood purifying apparatus, in which a flow controller controls a flow through a plurality of fluid chambers, according to an embodiment of the present invention; and

FIG. 20 is a view illustrating a flow controller according to an embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art. Terms or languages defined in the present disclosure may have different meaning according to the users' intention or practice. These terms should be interpreted as a meaning corresponding to the technical concept of the present invention disclosed throughout the specification of the present invention.

As used herein, the singular forms “a,” “an,” and “the” are intended to include the plural forms as well, unless the context clearly indicates otherwise. In addition, the expressions defining the relationship of elements or components should be interpreted as broad as possible. For example, it will be understood that when an element or layer is referred to as being “on,” “connected to,” “coupled to,” or “adjacent to” another element or layer, it can be directly on, connected, coupled, or adjacent to the other element or layer, or intervening elements or layers may be present therebetween.

It will also be understood that when an element is same or identical to another element, the element can be completely same or identical to another element, or it includes that the two elements may be “substantially” similar to each other. In the same manner, for the expression showing the equivalence of time such as “simultaneously” or “at the same time,” it should be understood that it happens completely at the same time, or they may happen at substantially the similar time.

The same reference denotations may be used to refer to the same or substantially the same elements throughout the specification and the drawings.

Hereinafter, a blood purifying apparatus according to embodiments of the present invention will be described in detail with reference to the accompanying drawings.

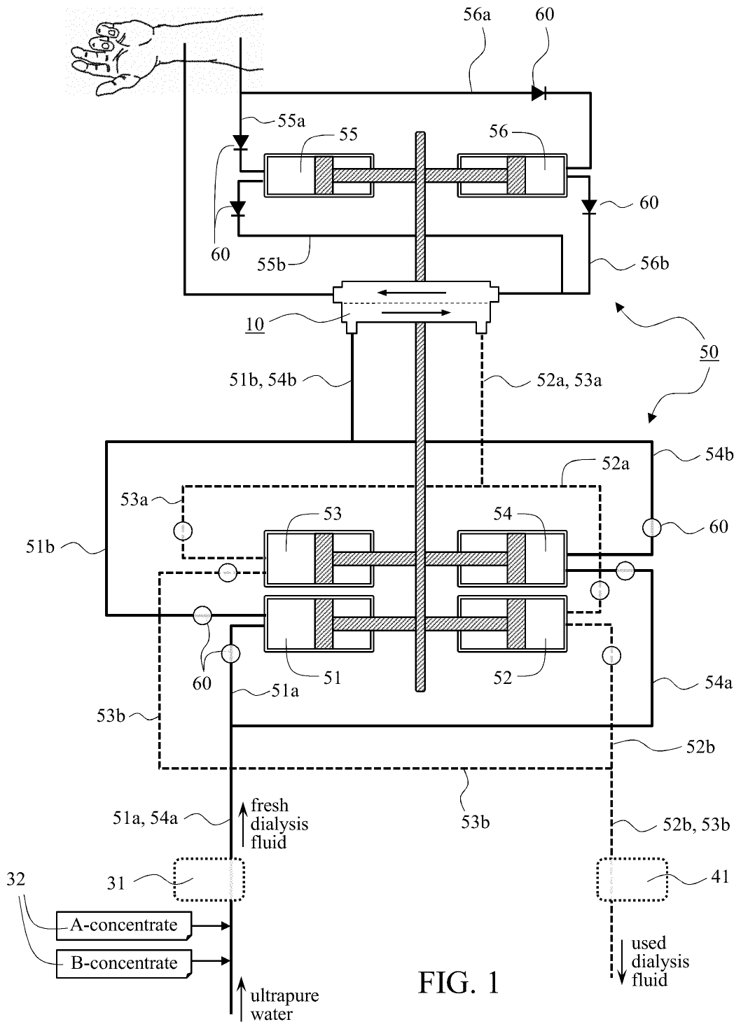

FIG. 1 is a schematic diagram of a blood purifying apparatus 1 according to an embodiment of the present invention. The blood purifying apparatus 1 shown in FIG. 1 is the device for hemodialysis to treat patients with end-stage renal disease (ESRD). However, the blood purifying apparatus 1 is not limited to the hemodialysis device and can include various devices. For example, the blood purifying apparatus 1 according to an embodiment of the present invention may include a device for liver dialysis for patients with acute or acute-on-chronic liver failure, a extracorporeal life supporter (ECLS) device for replacing impaired functions of a lung and/or a heart, or a device for treating patients with multiple organ failure requiring detoxification.

As shown in FIG. 1, the blood purifying apparatus 1 according to an embodiment of the present invention may include a blood purifying filter 10 in which mass transfer occurs between blood and dialysis fluid and a fluid pumping device 50 transferring the blood and dialysis fluid.

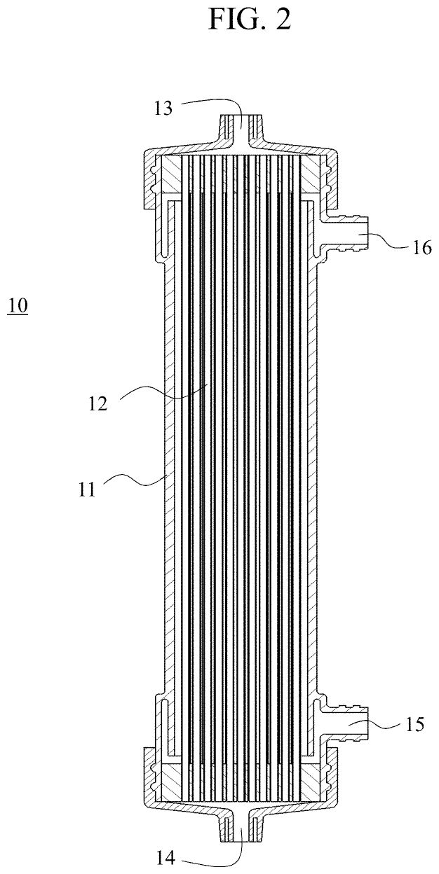

The blood purifying filter includes various filter apparatuses to purify blood. FIG. 2 illustrates an exemplary blood purifying filter 10. The blood purifying filter 10 according to an embodiment of the present invention may include a blood purifying filter container 11 having an internal space and a blood purifying membrane 12 accommodated in the internal space of the blood purifying filter container 11. The internal space of the blood purifying filter container 11 may be divided into a blood flow region and a dialysis fluid flow region by the blood purifying membrane 12. The blood purifying filter container 11 includes a first blood port 13 disposed at one end thereof and a second blood port 14 disposed at the other end thereof. Also, a first dialysis fluid port 15 and a second dialysis fluid port 16 may be provided on the outer surface of the blood purifying filter container 11 to allow the dialysis fluid to flow through the blood purifying filter 10. Blood passes through the blood flow region inside the blood purifying filter 10 and dialysis fluid passes through the dialysis fluid flow region. In this case, blood and dialysis fluid may be desirably configured to flow in the opposite directions to each other. The blood purifying filter 10 according to an embodiment of the present invention is not limited to the structure shown in the drawing, and may be modified into other forms, including but not limited to a hemodialyzer, an adsorption filter column, or a hemodiafilter.

The blood purifying apparatus 1 according to an embodiment of the present invention may include a dialysis fluid processing unit 32 through which the dialysis fluid is produced to be then provided to the blood purifying filter 10 for the blood purifying treatment. For example, the dialysis fluid processing unit 32 may be configured to mix acid and bicarbonate ion solutions (or acid and bicarbonate powder) with ultrapure water that is prepared through a water treatment unit. Through this process, ion concentrations such as bicarbonate, sodium, etc., and pH of the dialysis fluid can be adjusted within a predetermined level. However, the dialysis fluid is not limited to be produced through the aforementioned method and may be prepared in other ways. In an embodiment, the dialysis fluid may be provided by using a pre-made dialysis fluid bag.

Also, the blood purifying apparatus 1 according to an embodiment of the present invention may include a fresh dialysis fluid reservoir 31 to store fresh dialysis fluid and a used dialysis fluid reservoir 41 in which used dialysis fluid is collected. However, fresh dialysis fluid may be supplied to the blood purifying filter 10 without being stored in the fresh dialysis fluid reservoir 31 and the used dialysis fluid may be discarded without being collected in the used dialysis fluid reservoir 41.

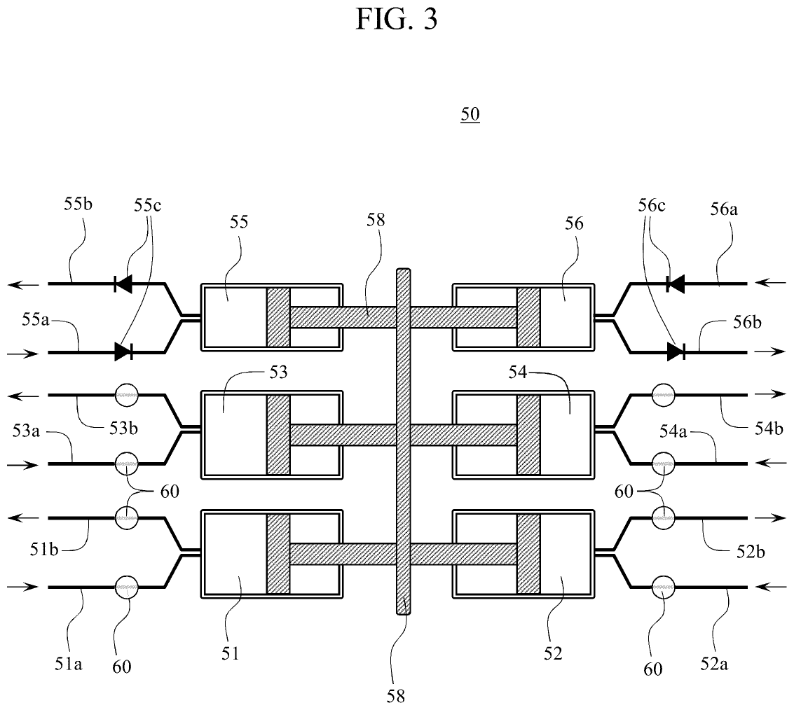

FIG. 3 is a view illustrating the fluid pumping device 50 according to an embodiment of the present invention. The fluid pumping device 50 may include a plurality of fluid chambers each having an internal space, a chamber pressurizing member 58 compressing or expanding the internal spaces of the fluid chambers, a pressurizing member driver (not shown) operating the chamber pressurizing member 58, and a flow controller 60.

The plurality of fluid chambers may further include first to six chambers 51 to 56 and each chamber is connected with a first flow tube through which a fluid flows into the chamber and a second flow tube through which a fluid contained in the chamber is discharged. The first and second flow tubes connected to each chamber can also be named an in-flow tube and an out-flow tube for convenience in description. For example, the first chamber 51 may be connected with a first chamber in-flow tube 51a and a first chamber out-flow tube 51b. A fluid flows into the first chamber 51 through the first chamber in-flow tube 51a and a fluid may be discharged from the chamber through the first chamber out-flow tube 51b. In the same manner, the second chamber 52 is connected with a second chamber in-flow tube 52a and a second chamber out-flow tube 52b. A fluid is supplied to the second chamber 52 through the second chamber in-flow tube 52a and is discharged from the second chamber 52 through the second chamber out-flow tube 52b. The same configuration can be applied to the third to sixth chambers 53 to 56.

Each of the chambers is connected with the in-flow and out-flow tubes. Here, the in-flow and out-flow tubes are merely expressions to describe the tubes connected to the chamber and it shouldn't be interpreted that a fluid must flow into the chamber through the in-flow tube and flow out of the chamber through the out-flow tube. For example, a fluid flows into the chamber through the out-flow tube, or a fluid may be provided to or discharged from the chamber through both the in-flow and out-flow tubes.

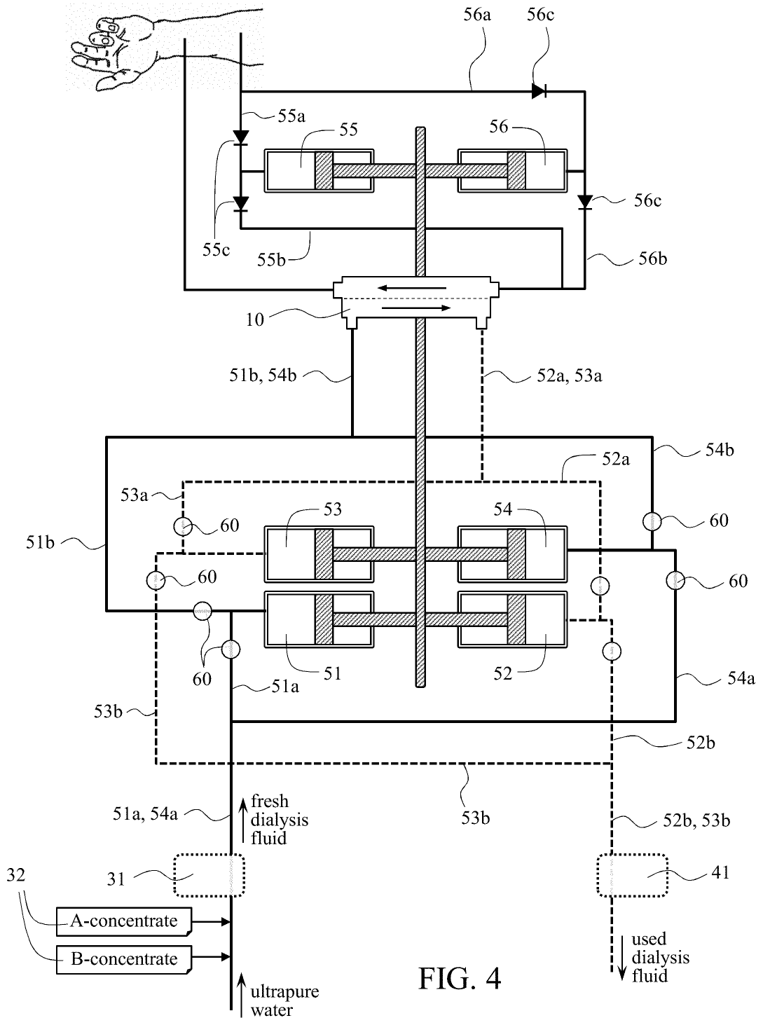

As shown in FIGS. 1 and 3, each chamber is connected with two tubes, i.e., in-flow and out-flow tubes. However, the in-flow and out-flow tubes may overlap in a portion such that a single tube is connected to the chamber as illustrated in FIG. 4.

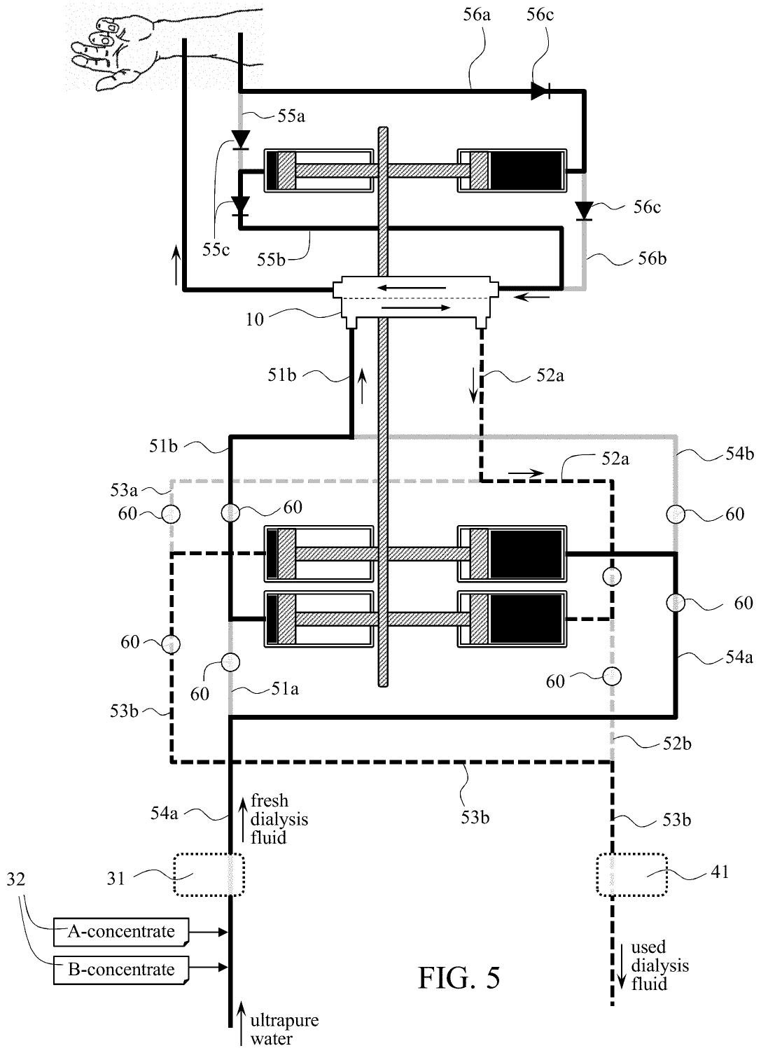

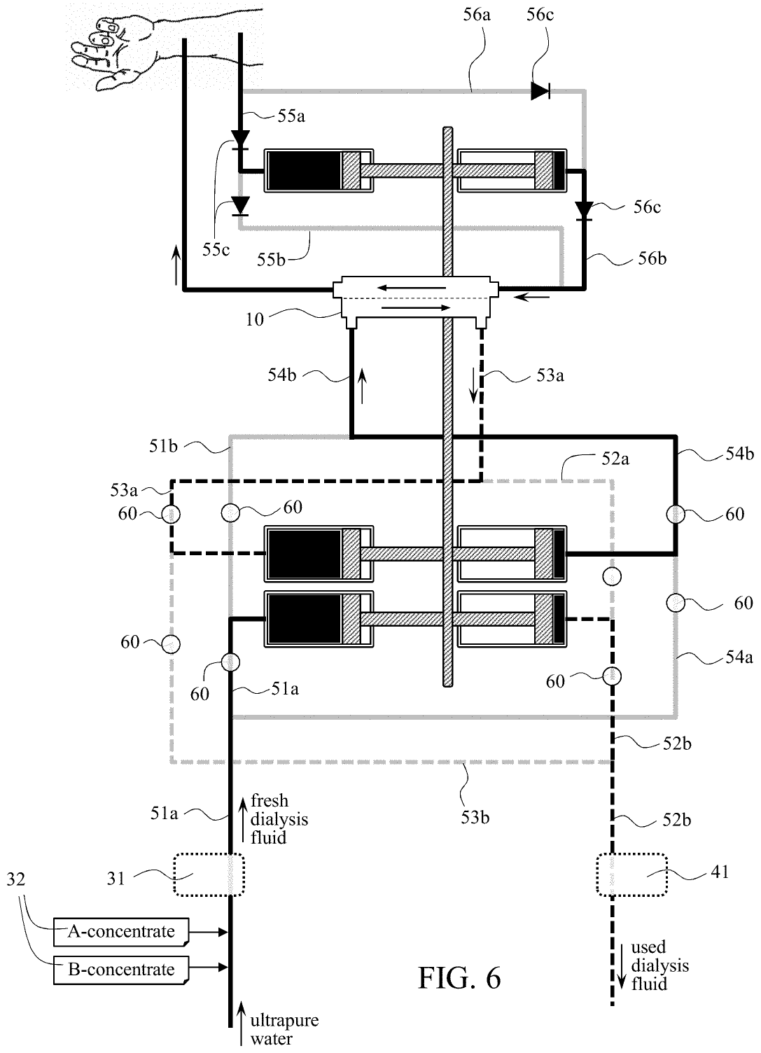

Due to the operation of the chamber pressurizing member 58, the chambers 51 to 56 may be compressed or expanded at the same time. This means that all of the chambers may be compressed simultaneously, or may be expanded simultaneously. Otherwise, any three chambers among the six chambers 51 to 56 may be expanded simultaneously while the other three chambers are expanded. As illustrated in FIGS. 5 and 6, when the chambers 51, 53 and 55 are compressed, the chambers 52, 54 and 56 are expanded, which occurs at the same time. This also means that when the chambers 51, 53 and 55 are expanded, the chambers 52, 54 and 56 are compressed.

As shown in FIGS. 3 to 6, the chambers are configured to have a cylinder-shaped internal space and the chamber pressurizing member 58 has a piston shape to easily compress and expand the internal spaces of the chambers. However, the blood purifying apparatus 1 is not limited to the structure shown in the drawings. For example, a container having an internal space to accommodate a fluid and any element which can pressurize or expand the internal space of the container to allow a fluid to flow through the container may be used for the chamber and chamber pressurizing member of the present invention. Exemplary fluid containers include a fluid sac, a fluid bag, or a fluid tube which may be made of a flexible material, and any element pressurizing or expanding the fluid sac, fluid bag or the fluid tube may be used for the chamber pressurizing member 58.

As aforementioned, since the chambers 51 to 56 are expanded and compressed simultaneously, a single chamber pressurizing member 58 may be used for compressing and expanding the chambers. In this regard, the blood purifying apparatus 1 according to an embodiment of the present invention may include a single chamber pressurizing member driver (now shown) to operate the chamber pressurizing member 58. The chamber pressurizing member driver may include various structures which allow the chamber pressurizing member 58 to reciprocate along a straight line or a curved line so as to compress or expand the internal spaces of the chambers. An exemplary chamber pressurizing member driver may include a cam pushing the chamber pressurizing member 58 in a rectilinear direction and a motor rotating the cam. Alternatively, the chamber pressurizing member driver may have a structure including a motor, a circular gear rotating by the motor, a linear gear moving along a straight line due to the rotation of the circular gear. Due to the rotation of the cam or circular gear, the chamber pressurizing member 58 moves along one direction, and when the motor rotates further or rotates in an opposition direction, the chamber pressurizing member 58 may move to the opposite direction.

The flow controller 60 controls a flow (or a flow passage) through the in-flow and out-flow tubes. In an embodiment, the flow controller 60 may regulate the flow through the in-flow and out-flow tubes connected to the first to fourth chambers 51 to 54. That is, the flow controller 60 controls a flow through the tubes 51a, 51b, 52a, 52b, 53a, 53b, 54a, and 54b. Specifically, the flow controller 60 may block the flow through the tubes 51a, 52b, 53a, 54b and the tubes 51b, 52a, 53b, 54a in an alternate manner. When the chambers 51 to 56 are compressed or expanded, the flow controller 60 may block the flow through at least four of the tubes 51a, 51b, 52a, 52b, 53a, 53b, 54a, and 54b.

The flow controller 60 can be modified so as to control the flow through the in-flow and out-flow tubes connected to the first to sixth chambers 51 to 56. That is, the flow controller 60 controls a flow through the tubes 51a, 51b, 52a, 52b, 53a, 53b, 54a, 54b, 55a, 55b, 56a, and 56b. Specifically, the flow controller 60 may block the flow through the tubes 51a, 52b, 53a, 54b, 55a, 56b and the tubes 51b, 52a, 53b, 54a, 55b, 56a in an alternate manner. In particular, when the chambers 51 to 56 are compressed or expanded, the flow controller 60 may block the flow through at least six among the tubes 51a, 51b, 52a, 52b, 53a, 53b, 54a, 54b, 55a, 55b, 56a, and 56b.

When the flow controller 60 controls a flow through the in-flow and out-flow tubes connected to the first to fourth chambers 51 to 54 (i.e., through the tubes 51a, 51b, 52a, 52b, 53a, 53b, 54a, and/or 54b), the blood purifying apparatus 1 according to an embodiment of the present invention may further include one-way valves installed on the flow tubes connected to the fifth chamber 55 and the sixth chamber 56; i.e., 55a, 55b, 56a and 56b. As shown in FIGS. 5 and 6, fifth chamber one-way valves 55c are disposed on the fifth chamber in-flow tube 55a and the fifth chamber out-flow tube 55b. Likewise, sixth chamber one-way valves 56c are disposed on the sixth chamber in-flow tube 56a and the sixth chamber out-flow tube 56b. The one-way valves installed on the corresponding tubes allow a fluid to flow in one direction, preventing a retrograde flow through the tubes. A cracking pressure of the one-way valve which opens a flow though the one-way valve may have various values according to the purpose of the blood purifying treatment.

FIGS. 5 and 6 are views illustrating the operation of the fluid pumping device 50 and the blood purifying apparatus 1 having the same according to an embodiment of the present invention.

In FIGS. 5 and 6, the dialysis fluid flows through the first to fourth chambers 51 to 54 and blood flows through the fifth and sixth chambers 55 and 56. Specifically, the dialysis fluid is supplied to the blood purifying filter 10 through the first chamber 51 and the fourth chamber 54, and the dialysis fluid of the blood purifying filter 10 is removed therefrom through the second chamber 52 and the third chamber 53. Thus, the first chamber 51 and the fourth chamber 54 may be connected to the first dialysis fluid port 15 of the blood purifying filter 10. The second chamber 52 and the third chamber 53 may be connected to the second dialysis fluid port 16 of the blood purifying filter 10. Also, the fifth chamber 55 and the sixth chamber 56 may be connected to either the first blood port 13 or the second blood port 14 of the blood purifying filter 10. In addition, since the dialysis fluid processing unit 32 produces the dialysis fluid which is then provided to the blood purifying filter 10, the dialysis fluid processing unit 32 may be connected to the first chamber 51 (through the first chamber in-flow tube 51a) and the fourth chamber 54 (through the fourth chamber in-flow tube 54a).

As shown in FIG. 5, the first chamber 51, the third chamber 53, and the fifth chamber 55 are compressed by the chamber pressurizing member 58 while the second chamber 52, the fourth chamber 54, and the sixth chamber 56 are expanded. At this time, the flow controller 60 blocks a flow passage through the first chamber in-flow tube 51a, the second chamber out-flow tube 52b, the third chamber in-flow tube 53a, and fourth chamber out-flow tube 54b, and opens a flow passage through first chamber out-flow tube 51b, the second chamber in-flow tube 52a, the third chamber out-flow tube 53b, and the fourth chamber in-flow tube 54a.

\textcircled{1} Due to the compression of the first chamber 51, the dialysis fluid of the chamber is provided to the blood purifying filter 10 through the first chamber out-flow tube 51b.

\textcircled{2} Due to the expansion of the second chamber 52, the dialysis fluid of the blood purifying filter 10 is supplied to the chamber 52 through the second chamber in-flow tube 52a.

\textcircled{3} Due to the compression of the third chamber 53, the dialysis fluid of the chamber is removed therefrom through the third chamber out-flow tube 53b.

\textcircled{4} Due to the expansion of the fourth chamber 54, the dialysis fluid is supplied to the chamber 54 through the fourth chamber in-flow tube 54a.

\textcircled{5} Due to the compression of the fifth chamber 55, blood of the chamber is supplied to the blood purifying filter 10 through the fifth chamber out-flow tube 55b.

\textcircled{6} Due to the expansion of the sixth chamber 56, blood of a patient is supplied to the sixth chamber 56 through the sixth chamber in-flow tube 56a.

Here, because of the one-way valves disposed on the flow tubes connected to the fifth chamber 55 and the sixth chamber 56, blood of the fifth chamber 55 may not flow backward to a patient and blood of the blood purifying filter 10 may not flow backward to the sixth chamber 56.

On the other hand, as shown in FIG. 6, the chambers 51, 53, and 55 are expanded by the chamber pressurizing member 58 while the chambers 52, 54, and 56 are compressed. The flow controller 60 opens a flow through the first chamber in-flow tube 51a, the second chamber out-flow tube 52b, the third chamber in-flow tube 53a, and fourth chamber out-flow tube 54b, and blocks a flow through first chamber out-flow tube 51b, the second chamber in-flow tube 52a, the third chamber out-flow tube 53b, and the fourth chamber in-flow tube 54a.

\textcircled{1} Due to the expansion of the first chamber 51, the dialysis fluid is supplied to the chamber through first chamber in-flow tube 51a.

\textcircled{2} Due to the compression of the second chamber 52, the dialysis fluid of the chamber is discharged therefrom through the second chamber out-flow tube 52b.

\textcircled{3} Due to the expansion of the third chamber 53, the dialysis fluid of the blood purifying filter 10 is supplied to the chamber through the third chamber in-flow tube 53a.

\textcircled{4} Due to the compression of the fourth chamber 54 to the dialysis fluid of the chamber is supplied to the blood purifying filter 10 through the fourth chamber out-flow tube 54b.

\textcircled{5} Due to the expansion of the fifth chamber 55, blood of a patient is supplied to the chamber through the fifth chamber in-flow tube 55a.

\textcircled{6} Due to the compression of the sixth chamber 56, blood of the chamber is supplied to the blood purifying filter 10 through the sixth chamber out-flow tube 56b.

Here, because of the one-way valves disposed on the flow tubes connected to the fifth chamber 55 and the sixth chamber 56, blood of the sixth chamber 56 may not flow backward to a patient and blood of the blood purifying filter 10 may not flow backward to the fifth chamber 55.

The black lines in FIGS. 5 and 6 indicate that there is a flow through the tubes and grey lines indicate no flow therethrough. For the flow tubes connected to the first to fourth chambers 51 to 54, straight lines indicate the flow tubes through which dialysis fluid is supplied to the blood purifying filter 10 and broken lines are the tubes through which dialysis fluid of the blood purifying filter 10 is discharged from the blood purifying filter 10.

As illustrated in the drawings, while the chambers are compressed or expanded, both blood and dialysis fluid can flow continuously through the blood purifying filter 10, which results in minimizing any damage to the blood vessel of the patient while achieving adequate blood purifying efficiency.

Exemplary operation of the blood purifying apparatus 1 is provided in FIGS. 5 and 6, in which the dialysis fluid flows through the first to fourth chambers 51 to 54 and blood flows through the fifth and sixth chambers 55 and 56. However, the blood purifying apparatus 1 according to an embodiment of the present invention is not limited thereto. For example, any fluid other than the blood and dialysis fluid such as plasma may flow through the chambers. In addition, the ‘first’ to ‘sixth’ chambers 51 to 56 are the expression merely for the convenience of the description. Hence, among the first to sixth chambers 51 to 56, the number of chambers through which a first fluid flows and the number of chambers through which a second fluid flows may be changed depending on the objective of the blood purifying treatment.

In order to regulate flowrates (i.e., the amount of a fluid flowing for a given time) through the chambers 51 to 56, the chambers 51 to 56 may have substantially the same stroke volume. The stroke volume of the chamber can be defined as a volume that is expanded or compressed by the chamber pressurizing member 58 when the chamber pressurizing member 54 moves left or right by a predetermined length in the drawings, e.g., FIGS. 5 and 6. When the chamber pressurizing member 58 moves left or right in a predetermined length, in order for the chambers to have substantially the same stroke volumes, the cross-sectional surface area of the internal space of the chamber may be same as or substantially similar to each other. When the internal space of the chamber is shaped into a cylinder, a radius of the cross-sectional surface of the internal space may be same as each other or substantially similar to each other.

Alternatively, the stroke volume of each chamber may be different from each other. In an embodiment, while the chambers 51 to 54 may have substantially the same stroke volume, the chambers 55 and 56 may be different from that of the chambers 51 to 54. For example, the stroke volume of the fifth and sixth chambers 55 and 56 may have approximately a half of the stroke volume of the chambers 51 to 54.

For the blood purifying apparatus 1 according to an embodiment of the present invention, the first chamber 51 and the fourth chamber 54 supply dialysis fluid to the blood purifying apparatus 1 in an alternate manner. When it is assumed that the stroke volume of the first and fourth chambers 51 and 54 is 30 ml and the chamber pressurizing member 58 repeats the expansion-compression cycle at 10 times per minute (10 cycles/min), 600 ml/min of dialysis fluid is supplied to the blood purifying filter 10. If the fifth chamber 55 and the sixth chamber 56 have a stroke volume of 18 ml, blood flows at 350 ml/min. In an embodiment, the stroke volume of the chamber and the frequency of expansion-compression cycle of the chamber pressurizing member 58 can be adjusted to regulate the flowrates through the blood purifying filter 10. The blood purifying apparatus 1 according to an embodiment of the present invention may be configured to have a blood flowrate of 100 to 600 ml/min and a dialysis fluid flowrate of 150 to 1,200 ml/min.

Hereinafter, the flow controller 60 according to an embodiment of the present invention will be described in detail with reference to the accompanying drawings.

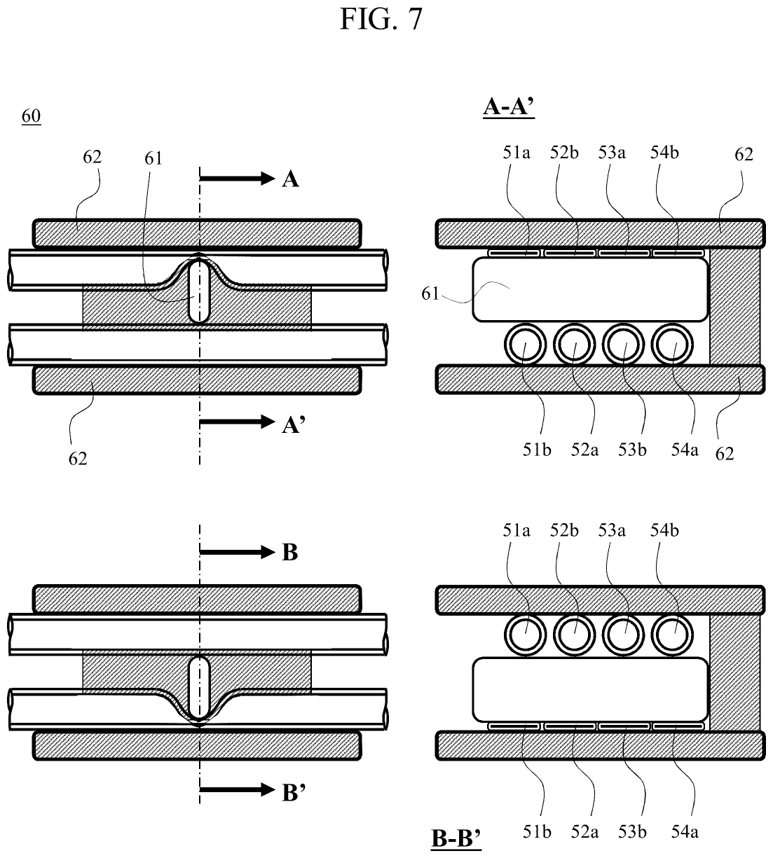

As aforementioned, the flow controller 60 according to an embodiment of the present invention controls a flow through the tubes 51a, 51b, 52a, 52b, 53a, 53b, 54a, and 54b, or the tubes 51a, 51b, 52a, 52b, 53a, 53b, 54a, 54b, 55a, 55b, 56a, and 56b. FIGS. 7 to 11 are views illustrating the flow controller 60 according to an embodiment of the present invention.

The flow controller 60 is formed of various structures. For example, the flow controller 60 may have a structure of a solenoid valve or an on-off valve which is installed on each of the flow tubes through which the flow controller controls a flow passage. The solenoid valve or the on-off valve can shut off the flow through the tubes.

Also, the flow controller 60 may have a structure of a pressurizing-type valve. The pressurizing-type valve may include a flow-blocking member compressing a portion of the flow tubes through which the flow controller controls a flow to block a flow therethrough, a flow-blocking wall supporting the flow tubes compressed by the flow-blocking member, and a flow-blocking member driver driving the flow-blocking member, such as by providing a liner or curved movement force to flow-blocking member.

The flow controller 60 in another embodiment may have a structure of a rotating-type valve, which includes a flow control housing having a cylinder-shaped internal space, a flow control rotor disposed inside the internal space of the flow control housing, a plurality of flow control ports each penetrating the flow control housing between the internal space and an outer surface thereof, and a rotor driver driving the flow control rotor.

In particular, the flow controller 60 according to an embodiment of the present invention may be configured to have any valve structure that is described above, or a combination thereof.

More specifically, FIG. 7 illustrates the flow controller 60 having a structure of the pressurizing-type valve. The flow controller 60 of FIG. 7 includes the flow blocking member 61 reciprocating in a straight line or in a curved line to compress a portion of the tubes 51a, 51b, 52a, 52b, 53a, 53b, 54a, or 54b, a flow-blocking wall 62 supporting the tubes 51a, 51b, 52a, 52b, 53a, 53b, 54a, or 54b compressed by the flow-blocking member 61, and a flow-blocking member driver (not shown) providing a straight or curved force to the flow-blocking member 61. When the flow-blocking member 61 moves to the tube 51a, 52b, 53a, and 54b, one end of the flow-blocking member 61 compresses the tubes supported by the flow-blocking wall 62 and blocks the flow therethrough. At this time, the flow passages through the tube 51b, 52a, 53b, and 54a are opened. Similarly, the flow-blocking member moves to the tubes 51b, 52a, 53b, and 54a, and another end of the flow-blocking member 61 compresses the tubes supported by the flow-blocking wall 62 and blocks the flow therethrough.

Here, although the flow-blocking member 61 is described to have an end and another end, the flow controller 60 is not limited to the structure. For example, the flow controller 60 may be able to control the flow through the tubes 51a, 51b, 52a, 52b, 53a, 53b, 54a, and/or 54b by using two or more flow-blocking members 61 which are separated from each other. In this case, two or more separate flow-blocking member drivers may be provided to operate each of the flow-blocking members 61.

Alternatively, when the tubes are made of flexible materials, such as silicone, polyurethane, polyacetate, etc., it may be possible to bend the flow tubes by a predetermined angle to thereby block the flow passage through the flow tubes. In other words, the flow-blocking member 61 may be able to not only compress the tubes to close the flow inside, but also bend the tubes to block the flow.

The flow-blocking member driver may include various structures that can apply a reciprocating movement force (that is, for a rectilinear or curvilinear movement) to the flow-blocking member 61. An exemplary flow-blocking member driver may include a cam for pushing the flow-blocking member 61 toward the flow-blocking wall 62 supporting the tubes and a motor rotating the cam. When the flow-blocking member 61 compresses the tubes due to the rotation of the cam, the flow therethrough may be blocked. When an external force by the cam is removed, the flow-blocking member 61 may detach from the tube, and the tube may be restored to the original state by its own elastic force, expanding the inside of the tube. Or, an eccentric cam connected to a motor may rotate and compress one side of the tube, and thus block the flow through the compressed tube. The cam further rotates such that an external force applied by the cam may be removed and the tube is restored to its original status, expanding the inside of the tube.

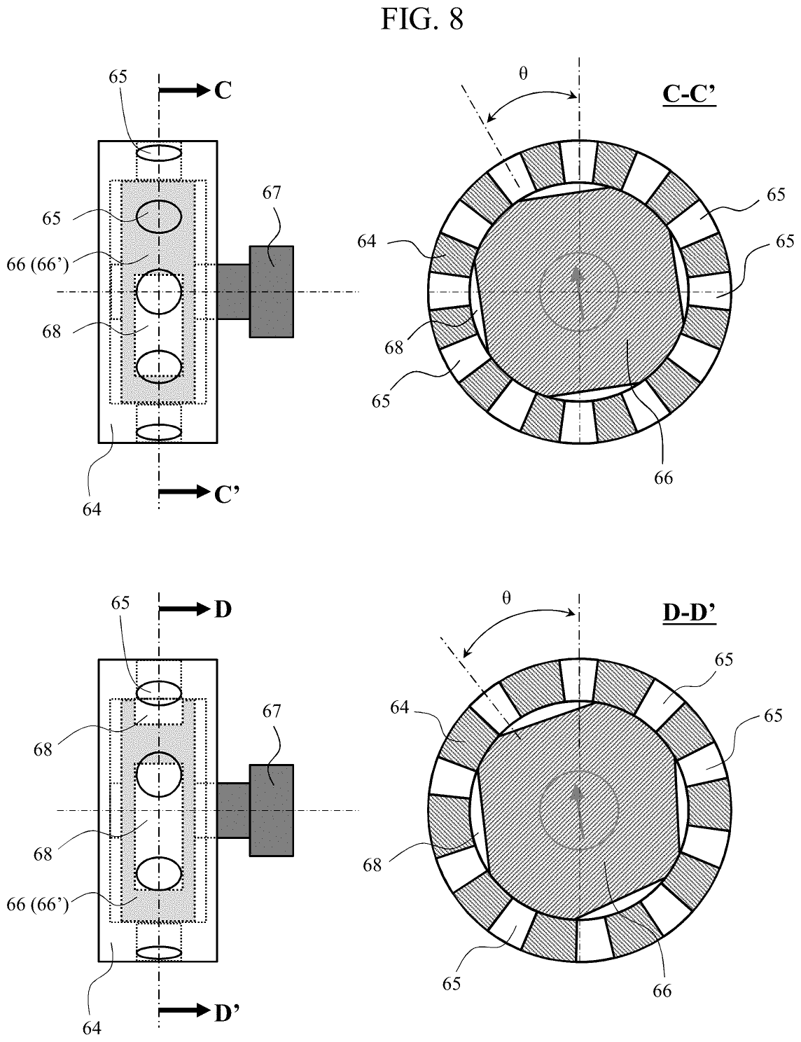

FIG. 8 is a view illustrating the flow controller 60 having a structure of the rotating-type valve. Thus, the flow controller 60 may include the flow control housing 64 having an internal space, the flow control rotor 66 which is disposed inside the flow control housing 64, the plurality of flow control ports 65 disposed on the flow control housing 64 and penetrating the flow control housing 64, and the rotor driver 67 operating the flow control rotor 66. In order for the flow control rotor 66 to rotate inside the flow control housing 64, the flow control rotor 66 and the internal space of the flow control housing 64 may have a cylindrical shape. However, the flow control rotor 66 is not limited to rotate inside the flow control housing 64 and may be modified to move along a rectilinear direction. Further, the flow control rotor 66 may be able to rotate while moving along a rectilinear direction. Due to the rotation or linear movement of the flow control rotor 66, a flow passage can be connected between two or more of the flow control ports 65.

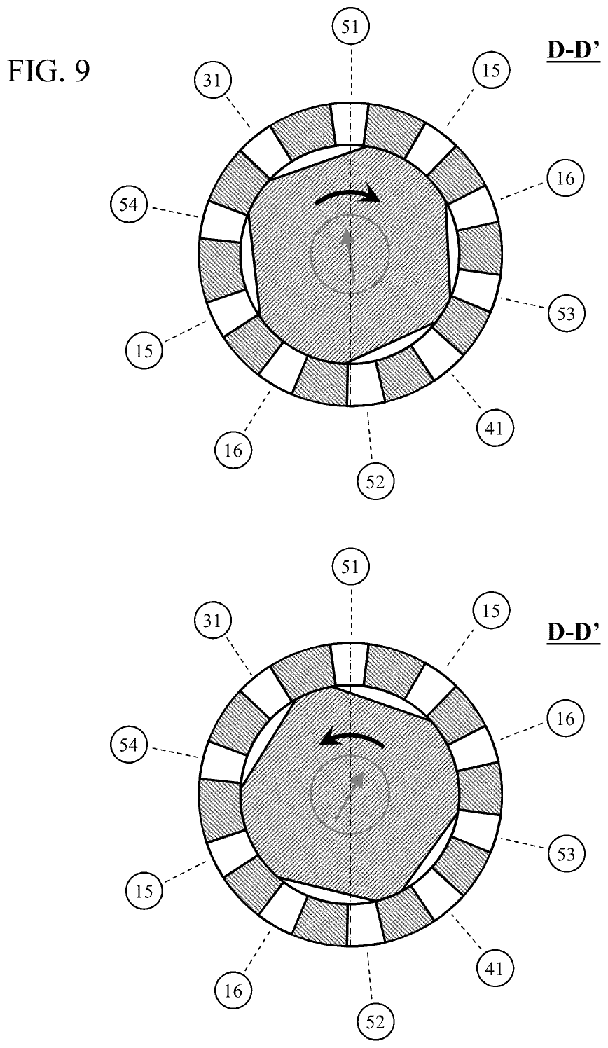

Here, the flow controller 60 may include 10 to 12 flow control ports 65. For example, as illustrated in FIG. 9, the 10 flow control ports 65 may be connected to the first chamber 51, first dialysis fluid port 15, second dialysis fluid port 16, third chamber 53, used dialysis fluid reservoir 41, second chamber 52, second dialysis fluid port 16, first dialysis fluid port 15, fourth chamber 54, and fresh dialysis fluid reservoir 31, respectively. Here, when the fresh dialysis fluid reservoir 31 is not used, the flow control port 65 connected to the fresh dialysis fluid reservoir 31 indicates that fresh dialysis fluid can be supplied through the port. Likewise, when the used dialysis fluid reservoir 41 is not used, the port 65 connected to the used dialysis fluid reservoir 41 indicates that used dialysis fluid may be discarded through the port.

In FIG. 9, when the flow control rotor 66 connects a flow passage between the ports connected to the first chamber 51 and the fresh dialysis fluid reservoir 31, respectively, dialysis fluid can be supplied to the first chamber 51 through the connected ports. That is, a flow passage is opened between the ports. However, the flow passage through the first dialysis fluid port 15 is blocked. In addition, as shown in FIG. 9 at the bottom, when the flow control rotor 66 further rotates or rotates in an opposition direction and connects a flow passage between ports connected to the first chamber 51 and the first dialysis fluid port 15, respectively, the dialysis fluid of the first chamber 51 may be supplied to the blood purifying filter 10 through these connected ports. However, the flow through the fresh dialysis fluid reservoir 31 is blocked. The same description can be applied to the flow control ports 65 connected to the second chamber 52, the third chamber 53, and the fourth chamber 54.

The flow control ports 65 formed in the flow control housing 64 may be spaced apart along a circumferential direction of the internal space of the flow control housing 64 which has a cylinder shape (or the flow control rotor 66 having a cylinder shape). In addition, the flow control ports 65 may be placed within substantially the same cross-sectional plane which is perpendicular to an axial direction of the internal space of the flow control housing 64. For example, the flow control ports 65 of FIG. 8 are placed within substantially the same cross-sectional plane of C-C′ or D-D′. In other words, the flow control ports 65 can be placed at substantially the similar elevation along an axial direction of the flow control rotor 66.

When the flow control ports 65 are placed within substantially the same plane, an angle θ between two adjacent flow control ports 65 with respect to the axial center of the flow control rotor 66 can be formed, as shown in FIG. 8. The angles between two adjacent flow control ports 65 with respect to the axial center of the flow control rotor 66 may be constant or may be varying. For example, the angle between two flow control ports 65 connected to the first chamber 51 and the fresh dialysis fluid reservoir 31, respectively, has a value of θ1, and the angle between two flow control ports 65 connected to the first chamber 51 and the first dialysis fluid port 15, respectively, may have a value of θ2.

In addition, as shown in FIG. 8, when the flow control rotor 66 is installed inside the flow control housing 64, one end of the flow control port 65 located in a side close to the inner surface of the flow control housing 64 may be placed within the cylindrical surface 66′ of the flow control rotor 66. More specifically, the end of the flow control port 65 located at the inner surface of the flow control housing 64 can be configured to face a middle portion of the flow control rotor 66 along an axial direction.

The flow control rotor 66 according to an embodiment of the present invention rotates unidirectionally or bidirectionally to control the opening and blocking of the flow passage through the flow control ports 65. However, as aforementioned, the flow control rotor 66 can move along a rectilinear direction or rotate while moving along a rectilinear direction. The time for opening or blocking the flow passage can be controlled by regulating the movement speed of the flow control rotor 66.

In addition, the flow control rotor 66 may be further formed with a recessed portion 68 to make it easier for a fluid to flow through two adjacent flow control ports 65. The recessed portion 68 has a cross-sectional shape of a crescent moon in FIG. 8, but the cross-sectional shape of the recessed portion 68 may be modified into other shapes such as rectangular, square, quadrilateral, or triangular shapes.

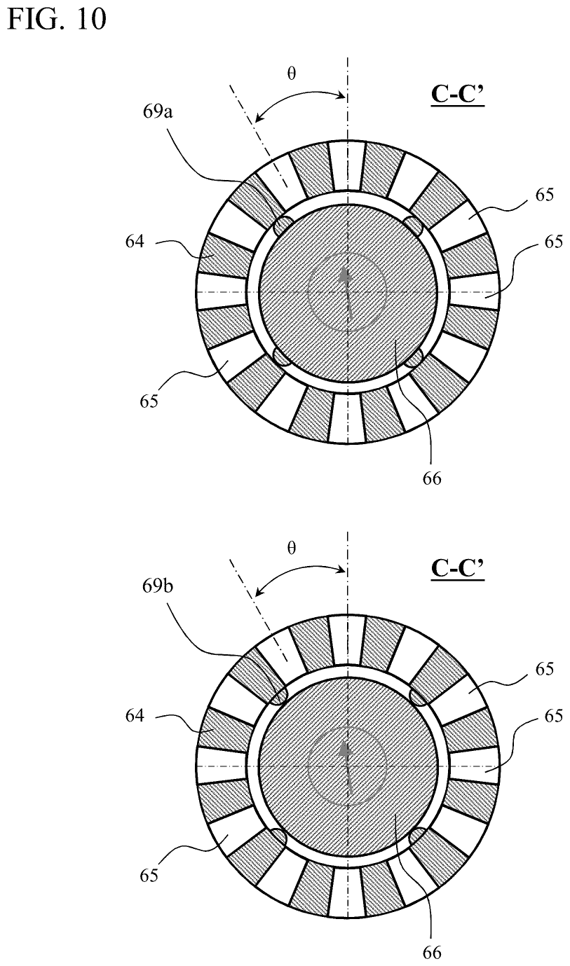

The flow control rotor 66 needs to be tightly attached to the inner surface of the flow control housing 64 to inhibit a fluid from leaking through the contact surface of the flow control rotor 66 and the flow control housing 64. In order to prevent the leakage of a fluid, the flow control rotor 66 and the flow control housing 64 can be made of materials that can prevent a fluid from passing through the contact surface such as polymer. In addition, in order to prevent a leakage of any fluid through the contact surface, the flow control rotor 66 may be provided with a protrusion 69a such as an o-ring or a gasket. The protrusion 69a can be made of a flexible material such as rubber or silicone, or a hard material such as metal, aluminum, plastic, polymer, and the like to efficiently prevent the fluid leakage. Alternatively, as shown in FIG. 10, a protrusion 69b may be formed on the inner surface of the flow control housing 64. The flow controller 60 may have a different structure to prevent a fluid leakage through the contact surface between the flow control rotor 66 and the flow control housing 64.

The flow controller 60 is not limited to the structures shown in FIGS. 7 to 10 or described in paragraphs [0047] to [0050]. The flow controller 60 may be modified into other structures that can open or close the flow passages through the tubes 51a, 51b, 52a, 52b, 53a, 53b, 54a, and 54b, or through the tubes 51a, 51b, 52a, 52b, 53a, 53b, 54a, 54b, 55a, 55b, 56a, and 56b.

For example, the flow controller 60 according to an embodiment of the present invention may include a one-way valve which is installed on each flow tube through which the flow controller controls a flow passage. The one-way valve forces a fluid to flow in one direction.

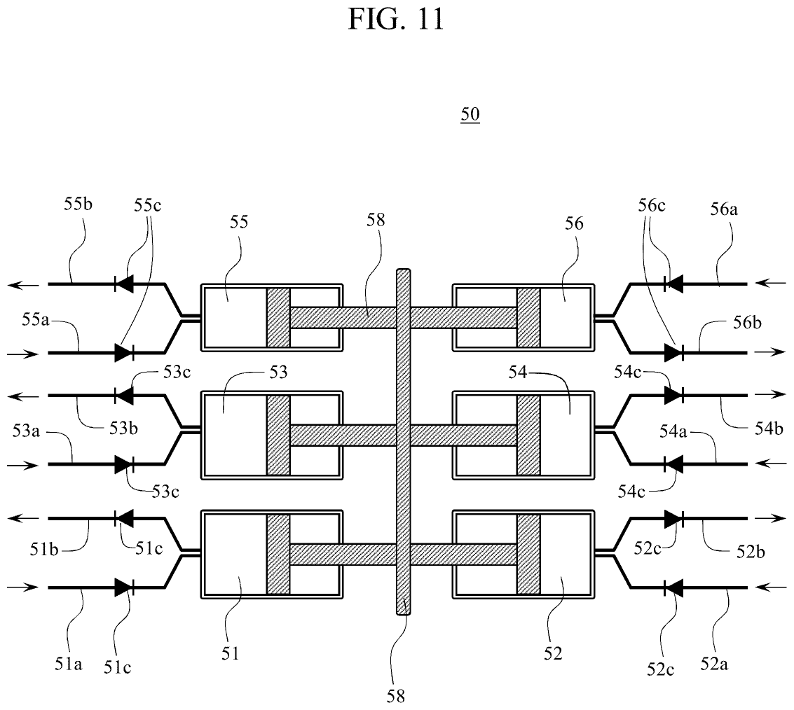

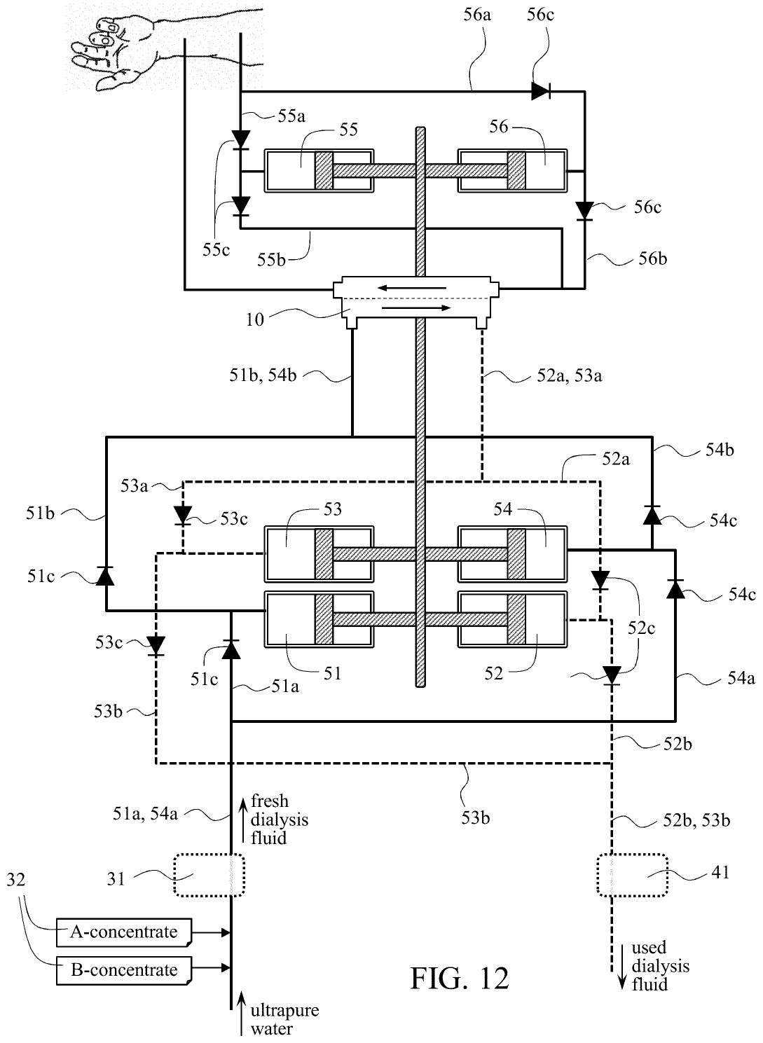

FIGS. 11 and 12 are views illustrating the fluid pumping device 50 and the blood purifying apparatus 1 in which the flow controller 60 is formed of the one-way valve (i.e., check valve) installed on each of the tubes. Specifically, a first chamber one-way valve 51c is installed on the first chamber in-flow tube 51a and the first chamber out-flow tube 51b. A second chamber one-way valve 52c is installed on the second chamber in-flow tube 52a and the second chamber out-flow tube 52b. A third chamber one-way valve 53c is installed on the third chamber in-flow tube 53a and the third chamber out-flow tube 53b. A fourth chamber one-way valve 54c is installed on the fourth chamber in-flow tube 54a and the fourth chamber out-flow tube 54b.

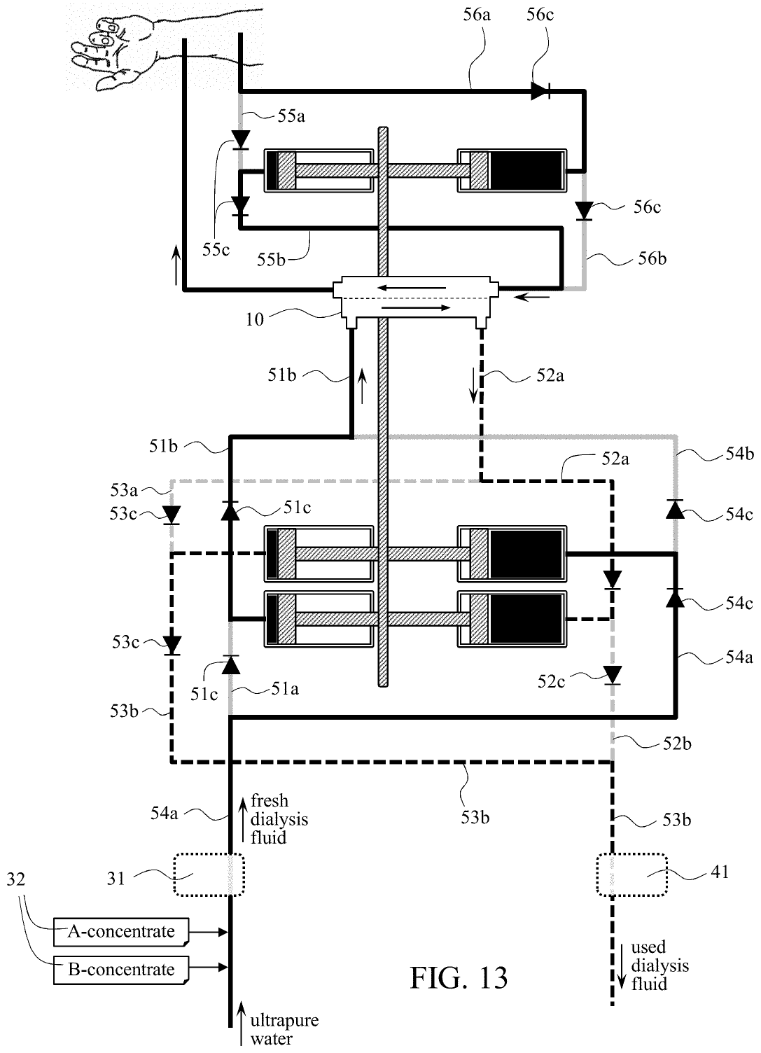

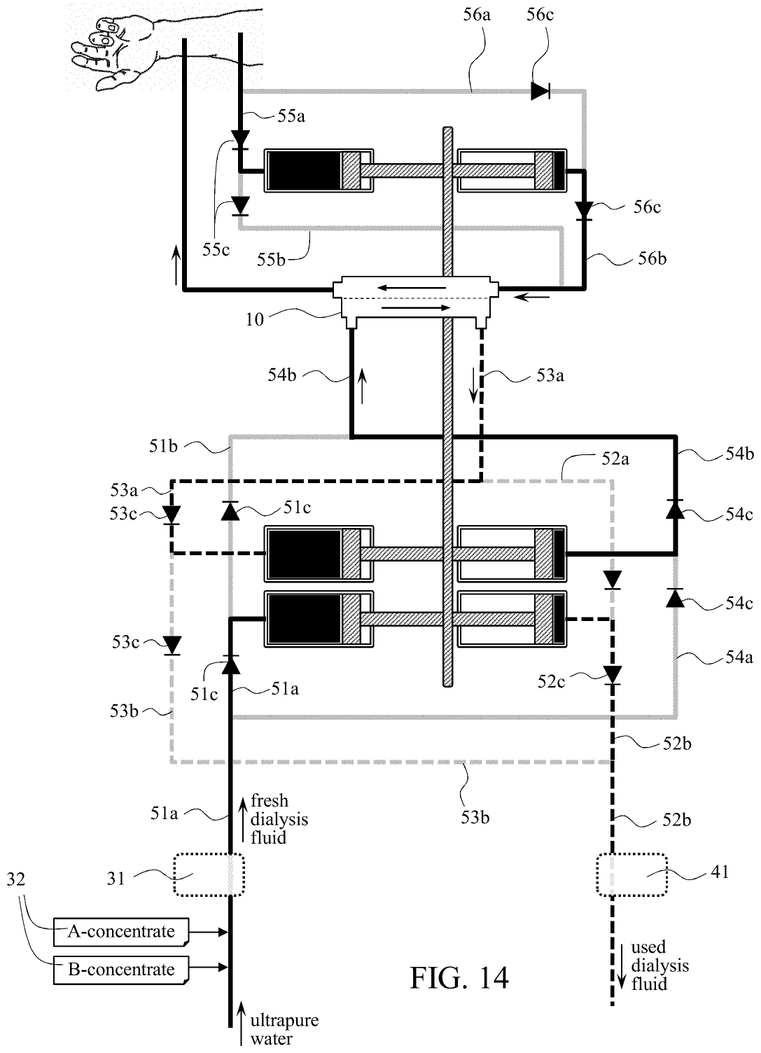

The operation of the blood purifying apparatus 1 where the one-way valves are provided in the tubes connected to the first to sixth chambers 51 to 56 are illustrated in FIGS. 13 and 14.

As shown in FIG. 13, the chambers 51, 53, and 55 are compressed while the chambers 52, 54, and 56 are expanded.

\textcircled{1} Due to the compression of the first chamber 51, the dialysis fluid of the chamber is provided to the blood purifying filter 10 through the first chamber out-flow tube 51b. Here, because of the first chamber one-way valve 51c installed on the first chamber in-flow tube 51a, the dialysis fluid may not flow backward to the direction of the fresh dialysis fluid reservoir 31 (i.e., through the first chamber in-flow tube 51a).

\textcircled{2} Due to the expansion of the second chamber 52, the dialysis fluid of the blood purifying filter 10 is supplied to the chamber 52 through the second chamber in-flow tube 52a. Here, because of the second chamber one-way valve 52c installed on the second chamber out-flow tube 52b, the dialysis fluid may not flow backward to the second chamber 52 through the second chamber out-flow tube 52b.

\textcircled{3} Due to the compression of the third chamber 53, the dialysis fluid of the chamber is removed therefrom through the third chamber out-flow tube 53b. Here, because of the third chamber one-way valve 53c installed on the third chamber in-flow tube 53a, the dialysis fluid may not flow backward to the blood purifying filter 10.

\textcircled{4} Due to the expansion of the fourth chamber 54, the dialysis fluid is supplied to the chamber 54 through the fourth chamber in-flow tube 54a. Here, because of the fourth chamber one-way valve 54c installed on the fourth chamber out-flow tube 54b, dialysis fluid of the blood purifying filter 10 may not flow backward to the chamber 54.

\textcircled{5} Due to the compression of the fifth chamber 55, blood of the chamber is supplied to the blood purifying filter 10 through the fifth chamber out-flow tube 55b.

\textcircled{6} Due to the expansion of the sixth chamber 56, blood of a patient is supplied to the sixth chamber 56 through the sixth chamber in-flow tube 56a.

Here, because of the one-way valves mounted on the tubes connected to the fifth chamber 55 and the sixth chamber 56, blood of the fifth chamber 55 may not flow backward to a patient and blood of the blood purifying filter 10 may not flow backward to the sixth chamber 56.

On the other hand, as shown in FIG. 14, the chambers 51, 53, and 55 are expanded while the chambers 52, 54, and 56 are compressed.

\textcircled{1} Due to the expansion of the first chamber 51, the dialysis fluid is supplied to the chamber through first chamber in-flow tube 51a. Here, because of the first chamber one-way valve 51c installed on the first chamber out-flow tube 51b, dialysis fluid of the blood purifying filter 10 may not flow backward to the chamber.

\textcircled{2} Due to the compression of the second chamber 52, the dialysis fluid of the chamber is discharged therefrom through the second chamber out-flow tube 52b. Here, because of the second chamber one-way valve 52c installed on the second chamber in-flow tube 52a, dialysis fluid may not flow backward to the blood purifying filter 10.

\textcircled{3} Due to the expansion of the third chamber 53, the dialysis fluid of the blood purifying filter 10 is supplied to the chamber through the third chamber in-flow tube 53a. Here, because of the third chamber one-way valve 53c installed on the third chamber out-flow tube 53b, the dialysis fluid may not flow backward to the chamber through the third chamber out-flow tube 53b.

\textcircled{4} Due to the compression of the fourth chamber 54 to the dialysis fluid of the chamber is supplied to the blood purifying filter 10 through the fourth chamber out-flow tube 54b. Here, because of the fourth chamber one-way valve 54c installed on the fourth chamber in-flow tube 54a, dialysis fluid may not flow backward to the direction of the fresh dialysis fluid reservoir 31 (i.e., through the fourth chamber in-flow tube 54a).

\textcircled{5} Due to the expansion of the fifth chamber 55, blood of a patient is supplied to the chamber through the fifth chamber in-flow tube 55a.

\textcircled{6} Due to the compression of the sixth chamber 56, blood of the chamber is supplied to the blood purifying filter 10 through the sixth chamber out-flow tube 56b.

Here, because of the one-way valves provided on the tubes connected to the fifth chamber 55 and the sixth chamber 56, blood of the sixth chamber 56 may not flow backward to a patient and blood of the blood purifying filter 10 may not flow backward to the fifth chamber 55.

The black lines in FIGS. 5 and 6 indicate that there is a flow through the tubes and grey lines indicate no flow therethrough. For the flow tubes connected to the first to fourth chambers 51 to 54, straight lines indicate the flow tubes through which dialysis fluid is supplied to the blood purifying filter 10 and broken lines are the tubes through which dialysis fluid of the blood purifying filter 10 is discharged from the blood purifying filter 10.

The one-way valves installed on the corresponding flow tubes allow a fluid to flow in one direction, preventing a retrograde flow through the tubes. A cracking pressure of the one-way valve which opens a flow though the one-way valve may have various values for the purpose of the blood purifying treatment. For example, when the flow controller 60 is not operating, the one-way valve may have a predetermined cracking pressure value which is enough to prevent a fluid from flowing through the one-way valves.

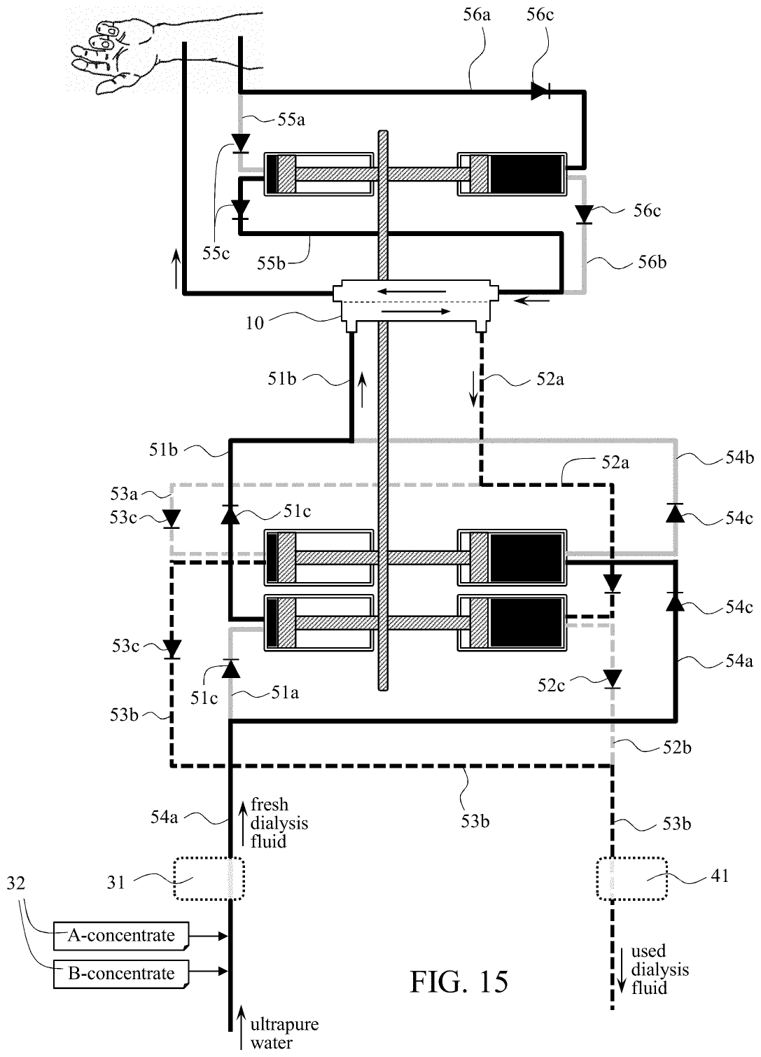

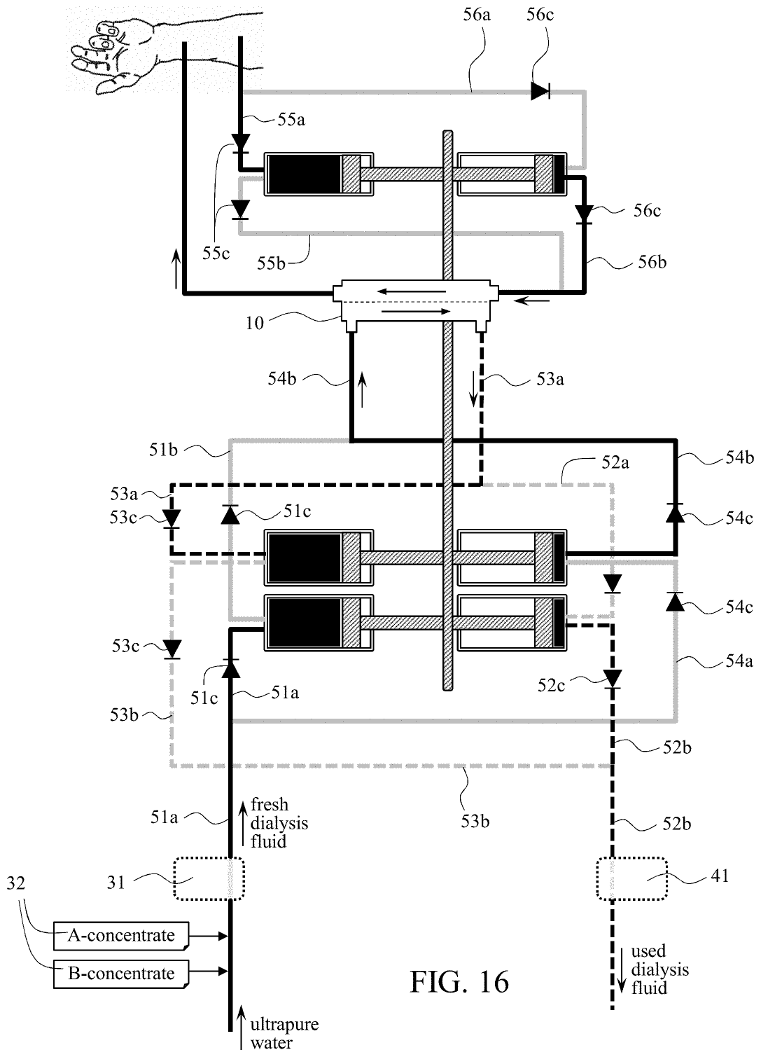

FIGS. 15 and 16 are views illustrating the operation of the blood purifying apparatus 1 in which the in-flow tube and the out-flow tube are separately connected to each chamber. The operation therefor is substantially same as that described in connection with FIGS. 13 and 14.

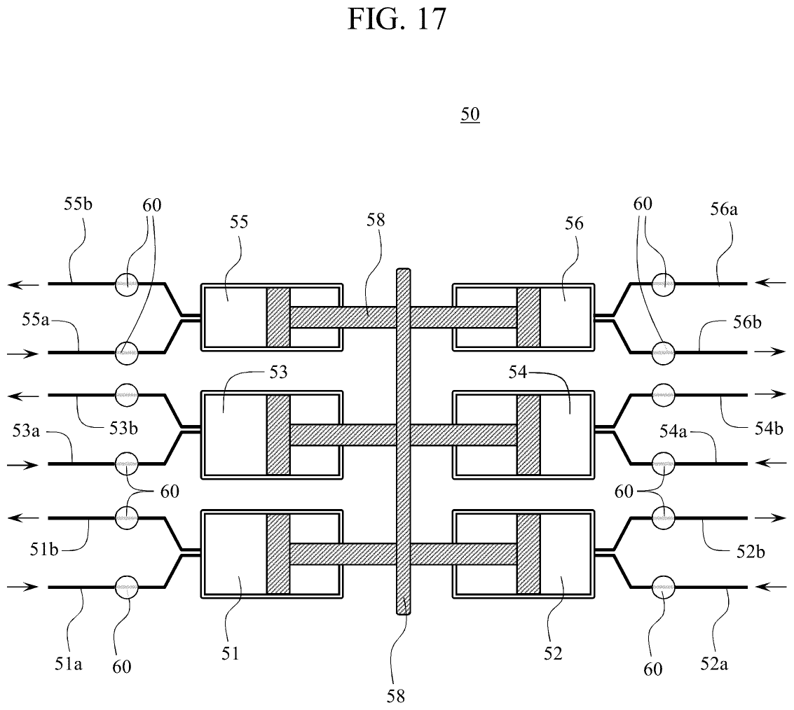

Again, the flow controller 60 can control the flow passages not only through the tubes 51a, 51b, 52a, 52b, 53a, 53b, 54a and 54b connected to the first to fourth chambers 51 to 54, but also the tubes 55a, 55b, 56a, and 56b connected to the fifth and sixth chambers 55 and 56. FIG. 17 is a view illustrating the flow controller 60 which can block the flow through the tubes 51a, 52b, 53a, 54b, 55a, 56b and the tubes 51b, 52a, 53b, 54a, 55b, 56a in an alternate manner. The flow controller 60 may have a various structure including a combination of two or more valve structures as described above and in the drawings. In particular, when the chambers 51 to 56 are compressed or expanded by the chamber pressurizing member 58, the flow controller 60 may be able to block the flow passages through at least a half of the tubes 51a, 51b, 52a, 52b, 53a, 53b, 54a, 54b, 55a, 55b, 56a, and 56b all the time.

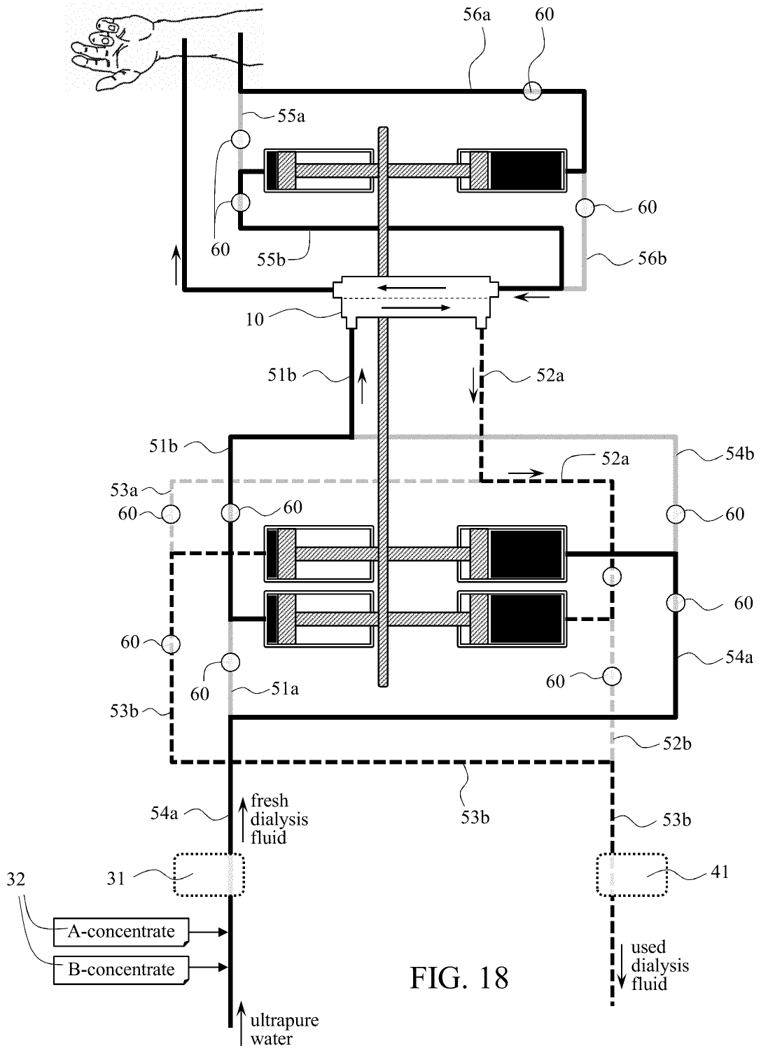

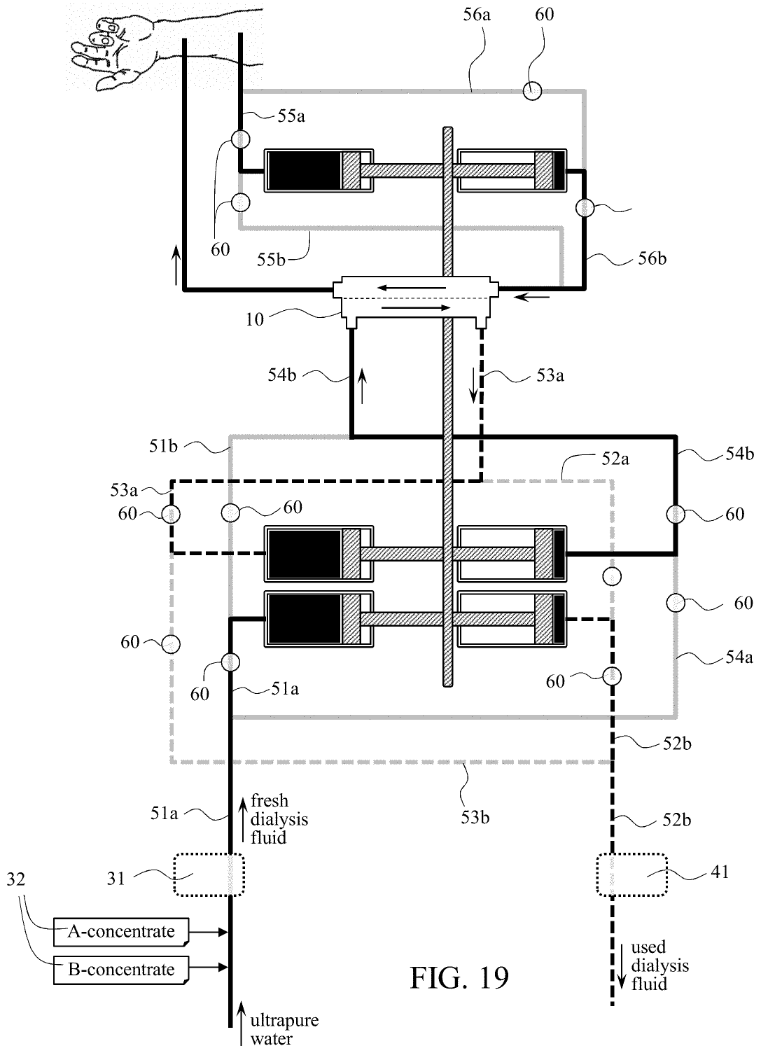

FIGS. 18 and 19 are views illustrating the operation of the blood purifying apparatus 1 where the flow controller 60 controls the flow through the 51a, 51b, 52a, 52b, 53a, 53b, 54a, 54b, 55a, 55b, 56a, and 56b. Except for the operation of the flow controller 60, substantially the same operation described in connection with FIGS. 5 and 6 may be applied to FIGS. 18 and 19.

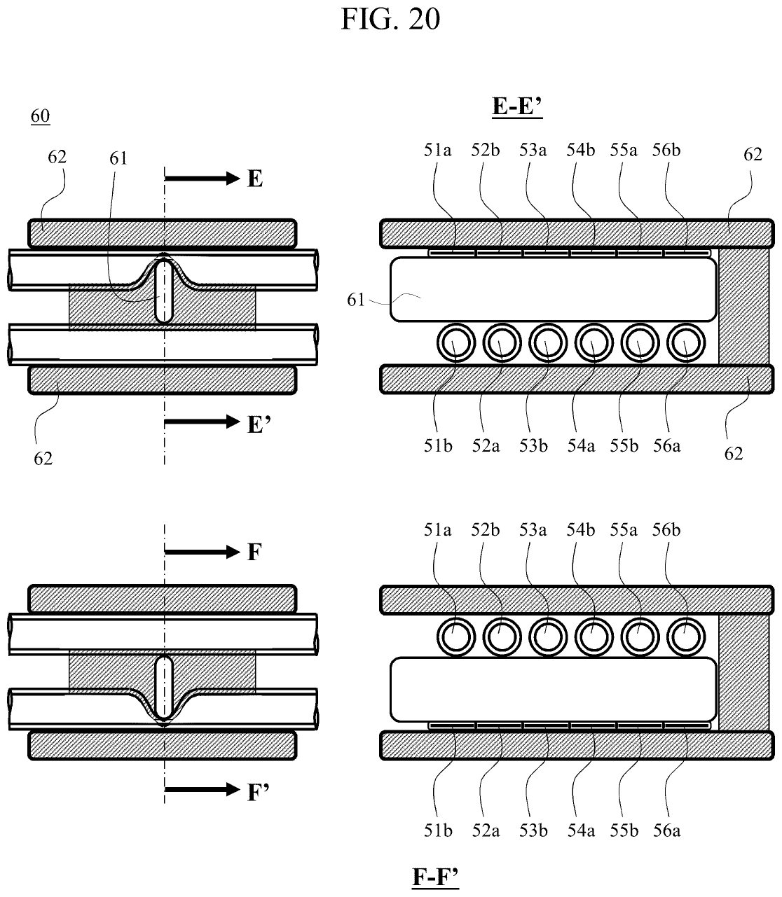

FIG. 20 is a view illustrating the exemplary flow controller 60 having a structure of a pressurizing-type valve, which further includes the flow-blocking member 61, the flow-blocking wall 62, and the flow-blocking member driver (not shown).

The blood purifying apparatus 1 according to an embodiment of the present invention may be able to compress or expand the plurality of fluid chambers, simultaneously, by using a single chamber pressurizing member to transfer blood and dialysis fluid together. Blood and dialysis fluid flow continuously through the blood purifying apparatus while the chambers are compressed and expanded, which permits blood purifying efficiency without causing a significant damage to the blood vessel of the patient.

Conventional blood purifying apparatuses, such as conventional hemodialysis devices, require a balancing chamber unit which is connected to both dialysis fluid supply and discharge lines, and two or more dialysis fluid pumps. In addition, blood is transferred by a separate blood pump. As such, the overall blood purifying apparatus becomes substantially complex in the structure and operation. It is also required to disinfect the balancing chamber unit and dialysis fluid pumps on a regular basis, which makes the unit further complicated for patients to use at home.

However, the blood purifying apparatus according to an embodiment of the present invention compresses and expands multiple fluid chambers using a single chamber pressurizing member, which removes the necessity of the disinfection procedure for the balancing chamber unit, dialysate pumps and the dialysis fluid tubes. Also, blood and dialysis fluid can be supplied continuously through the blood purifying filter and the entire system can be substantially simplified and easy to be installed while reducing the cost for blood purifying treatment. The blood purifying apparatus can also be easily installed and used by not only medical personnel but also patients (and their family members). Since a single driver can transfer both blood and dialysis fluid, cost for the device and treatment is substantially reduced and the overall device can be sufficiently miniaturized and light-weighted, making it suitable for an optimal alternative for blood purifying treatment in a place out of hospitals, such as at home.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A fluid pumping device comprising:
a plurality of fluid chambers each having an internal space;
a chamber pressurizing member compressing or expanding the internal spaces of the fluid chambers;
a chamber pressurizing member driver driving the chamber pressurizing member; and
a flow controller, wherein
the plurality of fluid chambers includes first to sixth chambers,
three chambers of the first to sixth chambers are compressed by the chamber pressurizing member while another three chambers are expanded,
each of the first to sixth chambers is connected with a first flow tube through which a fluid is provided to the chamber and a second flow tube through which a fluid of the chamber is discharged therefrom, and
the flow controller controls a flow through the flow tubes connected to the first to fourth chambers.

2. (canceled)

3. The fluid pumping device claim 1, further comprising a one-way valve installed on each of the flow tubes connected to the fifth chamber and the sixth chamber to allow a fluid to flow in one direction.

4. The fluid pumping device of claim 3, wherein the flow controller comprises at least one of:
a solenoid valve installed on each of the flow tubes through which the flow controller controls a flow to open or block a flow therethrough;
an on-off valve installed on each of the flow tubes through which the flow controller controls a flow to open or block a flow therethrough;
a pressurizing-type valve having a flow-blocking member compressing a portion of the flow tubes through which the flow controller controls a flow, a flow-blocking wall supporting the flow tubes compressed by the flow-blocking member, and a flow-blocking member driver providing a liner or curved movement force to the flow-blocking member; or
a rotating-type valve including a flow control housing having a cylinder-shaped internal space, a flow control rotor having a cylinder shape and disposed inside the internal space of the flow control housing, a plurality of flow control ports each penetrating the flow control housing between the internal space and an outer surface thereof, and a rotor driver driving the flow control rotor.

5. The fluid pumping device of claim 4, wherein when the chambers are compressed or expanded, the flow controller blocks a flow through at least a half of the flow tubes through which the flow controller controls a flow.

6. The fluid pumping device of claim 5, wherein when the flow controller includes the rotating-type valve, a flow passage through at least one of the flow control ports is blocked at any time, and
one end of the flow control ports facing the flow control rotor is placed within a cylinder surface of the flow control rotor.

7. The fluid pumping device of claim 1, wherein the flow controller further controls a flow through the flow tubes connected to the fifth chamber and the sixth chamber.

8. The fluid pumping device of claim 7, wherein the flow controller comprises at least one of:
a solenoid valve installed on each of the flow tubes through which the flow controller controls a flow to open or block a flow therethrough;
an on-off valve installed on each of the flow tubes through which the flow controller controls a flow to open or block a flow therethrough;
a pressurizing-type valve having a flow-blocking member compressing a portion of the flow tubes through which the flow controller controls a flow, a flow-blocking wall supporting the flow tubes compressed by the flow-blocking member, and a flow-blocking member driver providing a liner or curved movement force to the flow-blocking member; or
a rotating-type valve including a flow control housing having a cylinder-shaped internal space, a flow control rotor having a cylinder shape and disposed inside the internal space of the flow control housing, a plurality of flow control ports each penetrating the flow control housing between the internal space and an outer surface thereof, and a rotor driver driving the flow control rotor.

9. The fluid pumping device of claim 8, wherein when the chambers are compressed or expanded, the flow controller blocks a flow through at least a half of the flow tubes through which the flow controller controls a flow.

10. The fluid pumping device of claim 9, wherein when the flow controller includes the rotating-type valve, a flow passage through at least one of the flow control ports is blocked at any time, and one end of the flow control ports facing the flow control rotor is placed within a cylinder surface of the flow control rotor.

11. A blood purifying apparatus comprising:
a blood purifying filter in which mass transfer occurs between blood and dialysis fluid; and
a fluid pumping device according to claim 1 to transfer a biologic fluid, wherein
the blood purifying filter includes:
a blood purifying filter container having an internal space;
a first blood port provided in an end of the blood purifying filter container;
a second blood port provided in another end of the blood purifying filter container;
a first dialysis fluid port provided in one side of the blood purifying filter container; and
a second dialysis fluid port provided in another side of the blood purifying filter container.

12. The blood purifying apparatus of claim 11, wherein
a portion of the flow tubes connected to the fifth and sixth chambers is connected to the first blood port of the blood purifying filter,
a portion of the flow tubes connected to the first and fourth chambers is connected to the first dialysis fluid port of the blood purifying filter, and
a portion of the flow tubes connected to the second and third chambers is connected to the second dialysis fluid port of the blood purifying filter.

13. The blood purifying apparatus of claim 12, further comprising a one-way valve installed on each of the flow tubes connected to the fifth chamber and the sixth chamber to allow a fluid to flow in one direction.

14. The blood purifying apparatus of claim 13, wherein the flow controller comprises at least one of:
a solenoid valve installed on each of the flow tubes through which the flow controller controls a flow to open or block a flow therethrough;
an on-off valve installed on each of the flow tubes through which the flow controller controls a flow to open or block a flow therethrough;
a pressurizing-type valve having a flow-blocking member compressing a portion of the flow tubes through which the flow controller controls a flow, a flow-blocking wall supporting the flow tubes compressed by the flow-blocking member, and a flow-blocking member driver providing a liner or curved movement force to the flow-blocking member; or
a rotating-type valve including a flow control housing having a cylinder-shaped internal space, a flow control rotor having a cylinder shape and disposed inside the internal space of the flow control housing, a plurality of flow control ports each penetrating the flow control housing between the internal space and an outer surface thereof, and a rotor driver driving the flow control rotor.

15. The blood purifying apparatus of claim 14, wherein when the chambers are compressed or expanded, the flow controller blocks a flow through at least a half of the flow tubes through which the flow controller controls a flow.

16. The blood purifying apparatus of claim 15, wherein when the flow controller includes the rotating-type valve, a flow passage through at least one of the flow control ports is blocked at any time, and
one end of the flow control ports facing the flow control rotor is placed within a cylinder surface of the flow control rotor.

17. The blood purifying apparatus of claim 12, wherein the flow controller further controls a flow through the flow tubes connected to the fifth chamber and the sixth chamber.

18. The blood purifying apparatus of claim 17, wherein the flow controller comprises at least one of:
a solenoid valve installed on each of the flow tubes through which the flow controller controls a flow to open or block a flow therethrough;
an on-off valve installed on each of the flow tubes through which the flow controller controls a flow to open or block a flow therethrough;
a pressurizing-type valve having a flow-blocking member compressing a portion of the flow tubes through which the flow controller controls a flow, a flow-blocking wall supporting the flow tubes compressed by the flow-blocking member, and a flow-blocking member driver providing a liner or curved movement force to the flow-blocking member; or
a rotating-type valve including a flow control housing having a cylinder-shaped internal space, a flow control rotor having a cylinder shape and disposed inside the internal space of the flow control housing, a plurality of flow control ports each penetrating the flow control housing between the internal space and an outer surface thereof, and a rotor driver driving the flow control rotor.

19. The blood purifying apparatus of claim 18, wherein when the chambers are compressed or expanded, the flow controller blocks a flow through at least a half of the flow tubes through which the flow controller controls a flow.

20. The blood purifying apparatus of claim 19, wherein when the flow controller includes the rotating-type valve, a flow passage through at least one of the flow control ports is blocked at any time, and one end of the flow control ports facing the flow control rotor is placed within a cylinder surface of the flow control rotor.
